(19) United States
(12) Patent Application Publication
(10) Pub. No.: US 2013/0031645 A1
(43) Pub. Date: Jan. 31, 2013

(54) METHOD FOR HEPATIC DIFFERENTIATION OF DEFINITIVE ENDODERM CELLS

(71) Applicants: Thomas Touboul, Le Kremlin Bicetre (FR); Ludovic Vallier, Cambridge (GB); Anne Weber-Benarous, Le Kremlin Bicetre (FR)

(72) Inventors: Thomas Touboul, Le Kremlin Bicetre (FR); Ludovic Vallier, Cambridge (GB); Anne Weber-Benarous, Le Kremlin Bicetre (FR)

(21) Appl. No.: 13/511,726

(22) PCT Filed: Nov. 25, 2010
(86) PCT No.: PCT/EP10/68237

§ 371 Date: Oct. 18, 2012

(30) Foreign Application Priority Data
Nov. 25, 2009 (EP) 09306136.4

(51) Int. Cl. A61K 35/407; C12N 5/0735; A01K 67/027; C12N 5/0789; A61P 1/16; C12N 5/071; C12N 5/074

(52) U.S. Cl. 800/9; 435/377; 435/370; 435/325; 424/93.7

(57) ABSTRACT

The present invention relates to a method for obtaining a population of hepatic progenitor cells, said method comprising a step of culturing definitive endoderm cells with a culture medium stimulating hepatic specification. In a particular embodiment, such culture medium stimulating hepatic specification comprises a retinoic acid receptor (RAR) agonist, an FGF family growth factor and an inhibitor of the activin signaling pathway.

### FIELD OF THE INVENTION

The invention relates to a method for obtaining a population of hepatic progenitor cells, said method comprising a step of culturing definitive endoderm cells with a culture medium stimulating hepatic specification. In a particular embodiment, such culture medium stimulating hepatic specification comprises a retinoic acid receptor (RAR) agonist, an FGF family growth factor and an inhibitor of the activin signaling pathway.

### BACKGROUND OF THE INVENTION

Liver diseases are becoming one of the most common causes of mortality in developing countries. Orthotopic liver transplantation is currently the only available treatment. However an increasing number of patients die while on the liver transplant waiting list due to the shortage of suitable donor livers (Fox and Roy-Chowdhury, 2004). Hepatocyte transplantation recently became an alternative to orthotopic liver transplantation for the treatment of acute failure and life-threatening metabolic liver diseases (Puppi and Dhawan, 2009). However, this strategy is also restricted by the lack of donors and by the limited number of cells since functional human hepatocytes cannot be expanded in vitro and are difficult to cryopreserve. This group of diseases which targets hepatocytes which represent the dominant liver cells encompasses inherited metabolic disorders (such as Crigler-Najjar Syndrome type I, Glycogen storage disease, Urea cycle defects, familial hypercholesterolemia and tyrosinemia), chronic liver failure as well as acute liver failure, for which hepatocyte transplants can be infused as a bridge to organ transplantation. Therefore exploring other sources of cells to generate hepatic cells with the ability to proliferate in vitro and to express hepatic-specific functions remains a major goal.

Human embryonic stem cells (hES) and human induced pluripotent stem cells (hiPS) represent an advantageous source of cell for cell based therapy. Their capacity to self-renew confers upon them the capacity to grow almost indefinitely in vitro while maintaining the property to differentiate into a broad number of cell types including liver cells. Several groups have already reported the differentiation of hES cells into hepatocyte-like cells using diverse culture systems (Chiao et al., 2008; Duan et al., 2007; Lavon et al., 2004; Rambhatla et al., 2003; Schwartz et al., 2005; Shirahashi et al., 2004). However, these approaches are all based on culture media containing serum, complex matrices such as matrigel and animal products. All of which are source of unknown factors that could obscure analysis of developmental mechanisms or render the resulting tissues incompatible with future clinical applications. More importantly, the functionality of hepatocytes generated using these approaches remains to be demonstrated in vivo and the generation of fully differentiated hepatocytes in vitro still represent a major issue.

Therefore, there is still a need in the art for a method of obtaining at a high efficiency a population of hepatic progenitor cells from definitive endoderm cells. The finding of defined culture conditions for differentiating hepatic progenitor cells from definitive endoderm cells represents indeed a major step towards the generation of fully functional liver cells compatible with cell based therapy of liver diseases.

### SUMMARY OF THE INVENTION

The present invention relates to a method for obtaining a population of hepatic progenitor cells comprising a step of culturing definitive endoderm cells with a culture medium stimulating hepatic specification.

### DETAILED DESCRIPTION OF THE INVENTION

The inventors have developed a new culture system to drive differentiation of definitive endoderm cells into hepatic progenitor cells using fully defined culture system devoid of animal products or unknown factors which could impair the use of the resulting cells for cell based therapy. Importantly this approach follows a natural path of development by respecting key stages of liver development which may provide the best approach for generating differentiated cells with native properties. Thus, definitive endoderm cells can be differentiated into hepatic progenitors, which can then be matured further into fetal hepatocytes then fully differentiated hepatocytes showing functionality in vitro and in vivo.

### DEFINITIONS

As used herein, the term “definitive endoderm cells” refers to cells which typically express the following markers Sox17, GSC, Mix11, Lhx1, CXCR4, GATA6, Eomes and Hex. Moreover such definitive endoderm cells do not express extra-embryonic markers such as Sox7 and of neuroectoderm markers such as Sox2.

As used herein, the terms “hepatic progenitor cells”, “hepatoblasts” and “liver progenitors” are used herein interchangeably. They refer to cells that are capable of expressing characteristic biochemical markers, including but not limited to Alpha-fetoprotein (AFP), Albumine (Alb), Cytokeratin 19 (CK19) and Hepatocyte nuclear factor 4alpha (HNF4alpha). Such cells can differentiate into either foetal hepatocytes or into cholangiocytes and express markers of both lineages (i.e. as mentioned above CK19 which is a specific marker of cholangiocytes; HNF4 alpha and AFP which are specific markers of foetal hepatocytes).

As used herein, the term “foetal hepatocytes” refers to cells which are engaged in the hepatocytic lineage and which can give rise to mature hepatocytes. Typically, foetal hepatocytes express the following markers: Albumin, AFP, CK18, CK8, Apolipoprotein AII, Transtherythin, Alpha-1-antitrypsine, HNF4α, HNF3β, β1-integrine, c-Met, RLDL, Cyp3A7, ASGR. The capacity to internalize and to secrete indocyanine green is also typical of hepatocytes Moreover such foetal hepatocytes possess a cuboidal shape.

As used herein, the term “mature hepatocytes” or “liver cells” are used herein interchangeably. They refer to cells capable to uptake LDL, to store glycogen and secrete albumin and urea. Typically, mature hepatocytes express the following markers:aldolase B, albumin, Glut 4, TAT, TO, proteins for detoxification phase I: cytochrome P450, CYP 3A4, CYP 1A2, CYP 2B6, CYP 2C9, CYP 2E1, and proteins for detoxification phase II: BiIUGT as well as bile acid transporters.

As used herein, the terms “cholangiocytes”, “biliary cells”, “biliary epithelial cells” and “bile duct cells” are used herein interchangeably. They refer to the epithelial cells of the bile duct and contribute to bile secretion via release of phospholipids and biliary salts. Typically, cholangiocytes express the following markers: CK14, CK19, CK 7 and integrin β4.

As used herein, the term “pluripotent stem cells” refers to undifferentiated cells which have the potential to differentiate into any of the three germs layers: endoderm (interior stomach lining, gastrointestinal tract, the lungs), mesoderm (muscle, bone, blood, urogenital tractus), or ectoderm (epidermal tissues and nervous system). Pluripotent stem cells can thus give rise to any fetal or adult cell type. However, alone they cannot develop into a fetal or adult animal because they lack the potential to contribute to extraembryonic tissue, such as the placenta. Typically, pluripotent stem cells may express the following markers Oct4, Sox2, Nanog, SSEA 3 and 4, TRA 1/81, see International Stem Cell Initiative recommendations, 2007.

As used herein, the term “embryonic stem cells” or “ES cells” or “ESC” refers to cells that are pluripotent and have the ability to form any adult cell. ES cells are derived from fertilized embryos that are less than one week old. For example, human embryonic stem cells may be obtained according a protocol not involving the embryo destruction as described in (Chung et al., 2008; Revazova et al., 2007).

As used herein, the term “induced pluripotent stem cells” or “iPS cells” or “iPSCs” refers to a type of pluripotent stem cell artificially derived from a non-pluripotent cell (e.g. an adult somatic cell). Induced pluripotent stem cells are identical to embryonic stem cells in the ability to form any adult cell, but are not derived from an embryo. Typically, an induced pluripotent stem cell may be obtained through the induced ectopic expression of Oct3/4, Sox2, Klf4, and c-Myc genes in any adult somatic cell (e.g. fibroblast).

For example, human induced pluripotent stem cells (hiPS) may be obtained according to the protocol as described by (Takahashi et al., 2007; Yu et al., 2007) or else by any other protocol in which one or the other agents used for reprogramming cells in these original protocols are replaced by any gene or protein acting on or transferred to the somatic cells at the origin of the iPS lines. Basically, adult somatic cells are transduced with viral vectors, such as retroviruses, which comprise Oct3/4, Sox2, Klf4, and c-Myc genes.

The term “multipotent stem cells” as used herein refers to a stem cell that has the potential to give rise to cells from multiple, but a limited number of lineages.

For example, adult human stem cells that can be used in the methods of the present invention include but are not limited to, multipotent mesenchymal stromal cells (MSCs), adult multilineage inducible (MIAMI) cells (D'Ippolito et al., 2004; Reyes et al., 2002) (Reyes et al., 2002), MAPC (also known as MPC), cord blood derived stem cells (Kogler et al., 2004), and mesoangioblasts (Dellavalle et al., 2007; Sampaolesi et al., 2006). As used herein, the terms “multipotent mesenchymal stromal cells”, “mesenchymal stem cells” or “MSCs” are used herein interchangeably and refer to cells which are isolated mainly from bone marrow (Jiang et al., 2002) and adipose tissue (Aurich et al., 2009) (or fat tissue) but which have also been identified in other tissues such as synovium, periosteum or placenta. These cells are characterised by their property to adhere to plastic, their phenotype and their ability to differentiate into three different lineages (chondrocytes, osteoblasts and adipocytes).

As used herein, the term “culture medium” refers to any medium capable of supporting the growth and the differentiation of definitive endoderm cells into hepatic progenitor cells. Preferred media formulations that will support the growth and the differentiation of definitive endoderm cells into hepatic progenitor cells include chemically defined medium (CDM).

As used herein, the term “chemically defined medium” (CDM) refers to a nutritive solution for culturing cells which contains only specified components, preferably components of known chemical structure. A chemically defined medium is a serum-free and feeder-free medium.

As used herein, “serum-free” refers to a culture medium containing no added serum.

As used herein, “feeder-free” refers to culture medium containing no added feeder cells. The term feeder-free encompasses, inter alia, situations where definitive endoderm are passaged from a culture with feeders into a culture medium without added feeders even if some of the feeders from the first culture are present in the second culture.

Thus, a chemically defined medium is devoid of components derived from non-human animals, such as Foetal Bovine Serum (FBS), Bovine Serum Albumin (BSA), and animal feeder cells such mouse feeder cells.

Suitable CDM include humanised Johansson and Wiles CDM. Humanised Johansson and Wiles CDM is described in (Johansson and Wiles, 1995) and is supplemented with insulin, transferrin and defined lipids to which may be added polyvinyl alcohol (PVA) as substitute for Bovine Serum Albumin (BSA). As used herein, “CDM-PVA” refers to the humanised chemically defined medium of Johansson and Wiles comprising polyvinyl alcohol (PVA) instead of bovine or human serum albumin.

Thus, an appropriate CDM according to the invention may consist in 50% IMDM (e.g. from Invitrogen, Cergy, France) and 50% F12 NUT MIX (e.g. from Invitrogen), supplemented with 7 μg/ml of insulin (e.g. from Roche, Sandhofer, Germany), 15 μg/ml of transferrin (e.g. from Roche), 450 μM of monothioglycerol (e.g. from Sigma-Aldrich, St Quentin, France) and 1 mg/ml of Polyvinyl Alcohol (PVA; e.g. from Sigma).

As used herein, the expression “culture medium stimulating hepatic specification” refers to a culture medium that is capable of inducing the expression of hepatic markers such as Alpha-fetoprotein (AFP), Albumin (Alb) and Hepatocyte nuclear factor 4a (HNF4alpha).

As used herein, the term “marker” refers to a protein, glycoprotein, or other molecule expressed on the surface of a cell or into a cell, and which can be used to help identify the cell. A marker can generally be detected by conventional methods. Specific, non-limiting examples of methods that can be used for the detection of a cell surface marker are immunocytochemistry, fluorescence activated cell sorting (FACS), and enzymatic analysis.

As used herein, the term “nearly homogenous population” refers to a population of cells wherein the majority (e.g., at least about 60%, preferably at least about 70%, more preferably at least about 80%) of the total number of cells have the specified characteristics of the hepatic progenitor cells of interest.

A “receptor” or “receptor molecule” is a soluble or membrane bound/associated protein or glycoprotein comprising one or more domains to which a ligand binds to form a receptor-ligand complex. By binding the ligand, which may be an agonist or an antagonist, the receptor is activated or inactivated and may initiate or block pathway signalling.

A “receptor agonist” is a natural or synthetic compound which binds the receptor to form a receptor-agonist complex by activating said receptor and receptor-agonist complex, respectively, initiating a pathway signaling and further biological processes.

By “receptor antagonist” or “receptor inhibitor” is meant a natural or synthetic compound that has a biological effect opposite to that of a receptor agonist. The term is used indifferently to denote a “true” antagonist and an inverse agonist of a receptor. A “true” receptor antagonist is a compound which binds the receptor and blocks the biological activation of the receptor, and thereby the action of the receptor agonist, for example, by competing with the agonist for said receptor. An inverse agonist is a compound which binds to the same receptor as the agonist but exerts the opposite effect. Inverse agonists have the ability to decrease the constitutive level of receptor activation in the absence of an agonist.

As used herein, the term “pathologies” refers to any disease or condition associated with hepatic damage. The term “pathology associated with hepatic damage” refers to any disease or clinical condition characterized by hepatic damage, injury, dysfunction, defect, or abnormality. Thus, the term encompasses, for example, injuries, degenerative diseases and genetic diseases. In certain embodiments, pathologies of interest are genetic diseases including metabolic diseases, acute liver failure, chronic hepatitis

As used herein, the term “subject” refers to a mammal, preferably a human being, that can suffer from pathology associated with hepatic damage, but may or may not have the pathology.

In the context of the invention, the term “treating” or “treatment”, as used herein, refers to a method that is aimed at delaying or preventing the onset of a pathology, at reversing, alleviating, inhibiting, slowing down or stopping the progression, aggravation or deterioration of the symptoms of the pathology, at bringing about ameliorations of the symptoms of the pathology, and/or at curing the pathology.

### Methods for Obtaining Hepatic Progenitor Cells

In a first aspect, the present invention relates to a method for obtaining a population of hepatic progenitor cells comprising a step of culturing definitive endoderm cells with a culture medium stimulating hepatic specification.

Typically, said definitive endoderm cells are obtained from the differentiation of pluripotent or multipotent stem cells.

In one embodiment, the definitive endoderm cells are human definitive endoderm cells. In one preferred embodiment, said human definitive endoderm cells are obtained from pluripotent stem cells, such as human embryonic stem cells (ES) or human induced pluripotent cells (iPS), according to a method described in the international patent application WO 2008/056166. In particular, the definitive endoderm cells may be obtained by culturing ES or iPS for 1 to 4 days, preferably 2 days, in CDM-PVA supplemented with Activin 5-20 ng/ml, preferably 10 ng/ml, and FGF2 1-50 ng/ml, preferably 12 ng/ml; then for 1 to 5 days, preferably 3 days, in CDM-PVA supplemented with Activin 1-200 ng/ml, preferably 100 ng/ml, FGF2 1-100 ng/ml, preferably 20 ng/ml, BMP4 1-100 ng/ml, preferably 10 ng/ml, and LY294002 10 μM.

In another embodiment, said human definitive endoderm cells are obtained from multipotent stem cells such as umbilical cord blood stem cells.

In one embodiment, the pluripotent or multipotent stem cells contain a genetic mutation responsible for a hepatic genetic disease. Advantageously, in this embodiment, the population of hepatic progenitors cells obtained from said pluripotent or multipotent cells also contains said mutation and can therefore provide a good cellular model of the disease.

In one embodiment, the culture medium stimulating hepatic specification comprises an RAR agonist.

The term “RAR agonist” as used herein refers to any compound, natural or synthetic, which results in an increased activation of the retinoic acid receptor.

The retinoid receptors are indeed classified into two families, the retinoic acid receptors (RARs) and the retinoid X receptors (RXRs), each consisting of three distinct subtypes. Each subtype of the RAR gene family encodes a variable number of isoforms arising from differential splicing of two primary RNA transcripts. All-trans retinoic acid (ATRA) is the physiological hormone for the retinoic acid receptors and binds with approximately equal affinity to all the three RAR subtypes. ATRA does not bind to the RXR receptors and therefore is a selective RAR agonist. By “RAR selective agonist” it is meant a compound which activates RAR but which exhibits little or no activation of, or actually inhibits, RXR. In particular “selective” may denote that the affinity of the agonist for the retinoic acid receptors (RAR) is at least 25-fold, preferably 50-fold, more preferably 100-fold higher than the affinity for the retinoid X receptors (RXR).

Selectivity of an agonist for RAR may be assayed for instance by determining is said compound induces growth inhibition of a human head and neck squamous cell carcinoma (HNSCC) cell line, such as UMSCC10B, UMSCC11B, UMSCC14B, UMSCC17A, UMSCC17B, UMSCC22A, and UMSCC22B (Krause et al., 1981), UMSCC38 and 183A (Grenman et al., 1991), MDA886Ln (Sacks et al., 1989), 1483 (Sacks et al., 1988), SqCC/Y1 (Reiss et al., 1985), TR146 (Rupniak et al., 1985). It was indeed found that RAR-selective retinoids were active in inhibiting the growth of most of these HNSCC cell lines whereas RXR-selective agonists exhibited weak or no inhibitory effect on all these cell lines (Sun et al., 2000).

RAR or RXR selectivity may also be assayed by measuring receptor binding, transactivation activity and the ability to induce RXR homodimer formation as described in (38).

In one embodiment of the invention, the RAR agonist is a selective RAR agonist. According to this embodiment, the selective RAR agonists are selected in the group consisting of all-trans retinoic acid (RA), pan RAR agonists (i.e. compounds which activates the alpha, beta, and gamma isotypes of RAR) LGD 1550, E6060, selective RAR agonists such as CD336 (Am 580), AGN193312, Am555S, Am80, CD2314, AGN193174, LE540, CD437, CD666, CD2325, SR11254, SR11363, SR11364, AGN193078, TTNN (Ro 19-0645), CD270, CD271, CD2665, SR3985, AGN193273, Ch55, 2AGN190521, CD2366, AGN193109, Re80 (Sun et al., 1997). The RAR agonist may also be Ro 40-6976, Ro 13-7410 (TTNPB), Ro 11-0874, Ro 04-3780 (13-cis-RA), Ro 11-4824 (4-oxo-RA), Ro 11-1813, Ro 08-8717, Ro 10-0191, Ro 10-2655 (4-hydroxy-RA) and Ro 11-0976 (Crettaz et al., 1990), or Ro 40-6055 and Ro 41-5253 (Horn et al., 1996) or CD 2019.

In a preferred embodiment, the RAR agonist is all-trans retinoic acid (ATRA) which is the acid form of vitamin A.

The concentration of the RAR agonist in the culture medium stimulating hepatic specification may be from 10⁻⁸ M to 10⁻⁶ M, preferably about 10⁻⁷ M.

In another embodiment, the culture medium stimulating hepatic specification further comprises an FGF family growth factor and an inhibitor of the activin/nodal signaling pathway.

As used herein, the term “FGF family growth factor” refers to any naturally occurring substance (e.g. a protein) capable of stimulating cellular growth, proliferation and cellular differentiation by binding to one fibroblast growth factor receptor (FGFR). By binding to one FGFR, the substance increases for example the tyrosine phosphorylation of said receptor.

In one embodiment of the invention, the FGF family growth factor is selected from the group consisting of FGF7 (also known KGF), FGF10 and FGF22 which constitutes a subfamily (FGF7 subfamily (Yeh et al., 2003)) among FGF family members since these three growth factors preferably bind the keratinocyte growth factor receptor (KGFR) and the fibroblast growth factor receptor expressed by epithelial cells (FGFR2-IIb, and FGFR1B for FGF10 only).

In another embodiment, the FGF family growth factor is substance having a FGF10-like activity, e.g. a FGF10 mimetic. Such a substance may be identified by screening compounds for their capacity to restore FGF10 signaling in a cell knock-out for the receptor FGFR2b, or to activate FGFR2b at the surface of a cell contacted with an inhibitor of FGFR2b such as LPS, Pam₃Cys-Ser-(Lys)₄ and Sprouty (Spry) proteins.

In a preferred embodiment, the FGF family growth factor is FGF10. FGF10 can be purchased from AutogenBioclear. Typically, the FGF family growth factor, and in particular FGF10, is added to the culture medium of the invention in a concentration ranging from 1 to 200 ng/ml, preferably 20 to 100 ng/ml and preferably at about 50 ng/ml.

The term “inhibitor of the activin/nodal signaling pathway” as used herein refers to any compound, natural or synthetic, which results in a decreased activation of the activin/nodal signaling pathway, which is the series of molecular signals generated as a consequence of any member of the activin family binding to a cell surface receptor. Typically, an inhibitor of the activin/nodal signaling pathway provokes a decrease in the levels of phosphorylation of the protein Smad 2 (Shi and Massague, 2003).

The inhibitor of the activin/nodal signaling pathway may be an activin/nodal antagonist or a molecule which inhibits any downstream step of the activin/nodal signaling pathway. The inhibitor of the activin/nodal signaling may be a natural or a synthetic compound. When the inhibitor of the activin/nodal signaling pathway is a protein, it may be a purified protein or a recombinant protein or a synthetic protein.

Methods for producing recombinant proteins are known in the art. The skilled person can readily, from the knowledge of a given protein's sequence or of the nucleotide sequence encoding said protein, produce said protein using standard molecular biology and biochemistry techniques.

In one embodiment of the invention, the inhibitor of the activin/nodal signaling pathway is selected from the group consisting of SB431542, Lefty-A, Cerberus, Coco (accession number GenBank 22749329 or NCBI NP 689867.1) and derivatives of Lefty-A and Cerberus which inhibit the activin signaling pathway. Examples of such derivatives of Cerberus are truncated Cerberus (Cerb-S) (Smith et al, 2008), fragments of human Cerberus (accession number NCBI NP_{—}005445) which begin anywhere from residues 106-119 (inclusive) at the N-terminus and end anywhere after residue 241, and fragments of murin Cerberus (accession number NCBI NP_{—}034017) which begin anywhere from residues 106-119 (inclusive) at the N-terminus and end anywhere after residue 241.

In a preferred embodiment, the inhibitor of the activin/nodal signaling pathway is 4-(5-Benzol[1,3]dioxol-5-yl-4-pyrlidn-2-yl-1H-imidazol-2-yl)-benzamide hydrate also known as SB431542 which can be purchased from Tocris and Sigma. Typically, SB431542 is added to the culture medium of the invention in a concentration ranging from 1 to 100 μM, preferably 5 to 25 μM, still preferably at about 10 μM.

Preferably, the culture medium stimulating hepatic specification comprises The culture medium stimulating hepatic specification may comprise as a base medium CMD-PVA consisting of 50% IMDM and 50% F12 NUT, insulin 7 μg/ml, transferring 15 μg/ml, monothioglycerol 450 μM and Polyvinyl Alcohol (PVA) 1 mg/ml, supplemented with a RAR agonist, a FGF family growth factor, and an inhibitor of the activin/nodal signaling pathway, as described above.

Preferably, the culture medium stimulating hepatic specification comprises:
10⁻⁸ M to 10⁻⁶ M, preferably about 10⁻⁷ M of a RAR agonist, in particular ATRA;
1 to 200 ng/ml, preferably 20 to 100 ng/ml, still preferably about 50 ng/ml of a FGF family growth factor selected from the group consisting of FGF7, FGF10 and FGF22, preferably FGF10; and
1 to 100 μM, preferably 5 to 25 μM, still preferably about 10 μM SB431542.

Still preferably, the culture medium stimulating hepatic specification comprises a base medium CMD-PVA supplemented with:
10⁻⁸ M to 10⁻⁶ M, preferably about 10⁻⁷ M of a RAR agonist, in particular ATRA;
1 to 200 ng/ml, preferably 20 to 100 ng/ml, still preferably about 50 ng/ml of a FGF family growth factor selected from the group consisting of FGF7, FGF10 and FGF22, preferably FGF10; and
1 to 100 μM, preferably 5 to 25 μM, still preferably about 10 μM SB431542.

The step of culturing definitive endoderm cells with the culture medium stimulating hepatic specification shall be carried out for the necessary time required for the hepatic specification of definitive endoderm cells. The duration of this culture step may be determined easily by one of skill in the art. For instance, during the culture the person skilled in the art can monitor the cultured cells for the absence of expression of markers specifically expressed in definitive endoderm cells (e.g. Sox17, GSC, Mix11, Lhx1, CXCR4, GATA6, Eomes and Hex) and/or for the expression of markers specifically expressed by hepatic progenitor cells (e.g. Alpha-fetoprotein (AFP), Albumine (Alb), Cytokeratin 19 (CK19) and Hepatocyte nuclear factor 4alpha (HNF4alpha)). When expression of at least one, preferably several markers specific of definitive endoderm cells can not be detected and/or expression of at least one, preferably several markers specific of hepatic progenitor cells is detected, culturing with the culture medium stimulating hepatic specification can be stopped. Monitoring of these markers can be performed using for instance RT-PCR analysis of RNA extracted from cultured cells with specific primers, immunofluorescence analysis with antibodies specific of the markers and FACS, as shown in the examples below illustrating the invention. Typically, the culture of definitive endoderm cells with said medium stimulating hepatic specification may be carried out for at least 2 days, preferably at least 3 days, even more preferably at least 5 days. According to an embodiment, the culture of definitive endoderm cells with said medium stimulating hepatic specification is carried out for 2 to 5 days, in particular for 2 or 3 days.

The culture medium of the invention has to be renewed, partly or totally, at regular intervals. Typically, the culture medium of the invention can be replaced with fresh culture medium of the invention every other day.

The culture may be carried out in a support (plate, flask, etc) coated with a protein, peptide or molecule favouring cell adhesion, such as fibronectin, collagen or gelatine.

In one preferred embodiment, the definitive endoderm cells are previously cultured with an FGF family growth factor before culturing them with a culture medium stimulating hepatic specification. Thus, in this embodiment, definitive endoderm cells are cultured in a first step a) with an FGF family growth factor, then in a second step b) the cells cultured in step a) are cultured with said culture medium stimulating hepatic specification.

According to this embodiment, the FGF family growth factor is FGF10. Typically, FGF10 is added to the culture medium of the invention in a concentration ranging from 1 to 100 ng/ml, preferably at about 50 ng/ml.

Typically, the culture of definitive endoderm cells with said FGF family growth factor may be carried out for at least 2 days, preferably at least 3 days, even more preferably at least 5 days. For instance, the culture of definitive endoderm cells with said FGF family growth factor may be carried out for 2 to 10 days, preferably 2 to 5 days, still preferably for 3 days. The culture medium of the invention may be renewed, partly or totally, at regular intervals (e.g. every day).

The hepatic progenitor cells produced by the above method may be isolated and/or purified using any suitable method, for example flow cytometry.

The hepatic progenitor cells may, for example, be expanded or propagated in culture or used in clinical applications.

In some embodiments, hepatic progenitor cells may be further genetically modified with a nucleic acid of interest. Thus, the modified hepatic progenitor cells may be useful as vector for delivering acid nucleic.

In others embodiments, hepatic progenitor cells may be further differentiated.

The population of hepatic progenitor cells derived from definitive endoderm cells of the invention may be thus suitable for obtaining foetal hepatocytes.

### Methods for Obtaining Foetal Hepatocytes

Therefore, a second aspect of the invention relates to a method for obtaining a population of foetal hepatocytes comprising the steps of:
a. producing a population of hepatic progenitor cells according to a method of the invention, and
b. differentiating said population of hepatic progenitor cells into foetal hepatocytes.

In one preferred embodiment, the step of differentiating the population of hepatic progenitor cells into foetal hepatocytes is carried out by culturing said hepatic progenitor cells with a culture medium comprising an FGF family growth factor, an agonist of the EGF signaling pathway and an agonist of the HGF signaling pathway.

The term “FGF family growth factor” as used in connection with the method for obtaining foetal hepatocytes refers to any refers to any naturally occurring substance (e.g. a protein) capable of stimulating cellular growth, proliferation and cellular differentiation by binding to one fibroblast growth factor receptor (FGFR). By binding to one FGFR, the substance increases for example the tyrosine phosphorylation of said receptor.

In one embodiment, the FGF family growth factor is FGF4 (also known as heparin secretory transforming protein 1 or Kaposi sarcoma oncogene). Typically, FGF4 is added to the culture medium of the invention in a concentration ranging from 1 to 100 ng/ml, preferably 1 to 50 ng/ml, still preferably at about 30 ng/ml. FGF4 can be purchased from Peprotech.

The term “agonist of the EGF signaling pathway” as used herein refers to any compound, natural or synthetic, which results in an increased activation of the epidermal growth factor receptor (EGFR) which is the cell membrane receptor for EGF. The EGFR also binds other ligands that contain amino acid sequences classified as the EGF-like motif. The EGFR is also known as the ErbB-1 receptor and belongs to the type I family of receptor tyrosine kinases. A method for designing agonists to EGF receptor is for example described in international patent WO 99/62955.

In one embodiment of the invention, the agonist of the EGF signaling pathway is selected from the group consisting of epidermal growth factor (EGF), heparin-binding EGF-like growth factor (HB-EGF), vascular endothelial growth factor (VEGF) and Immunoglobulin-Binding Protein (IGBP).

In one preferred embodiment, the agonist of the EGF signaling pathway is EGF. Typically, EGF is added to the culture medium of the invention at a concentration ranging from 1 to 100 ng/ml, preferably at about 50 ng/ml. EGF can be purchased from Peprotech.

The term “agonist of the HGF signaling pathway” as used herein refers to any compound, natural or synthetic, which is capable of, directly or indirectly, substantially inducing, promoting or enhancing HGF biological activity or HGF receptor activation. HGF biological activity may, for example, be determined in an in vitro or in vivo assay of hepatocyte growth promotion as described in U.S. Pat. No. 6,099,841.

The agonist of the HGF signaling pathway may be hepatocyte growth factor (HGF) (Michieli et al., 2002) or any substance capable of activating HGF pathway, such as a drug, a synthetic or natural analog of HGF, for instance a truncated form of HGF. In particular the agonist may be magic-factor 1, a partial agonist of the Met tyrosine kinase, the high affinity receptor of HGF (39)

In one preferred embodiment, the agonist of the HGF signaling pathway is HGF. Typically, HGF is added to the culture medium of the invention in a concentration ranging from 1 to 100 ng/ml, preferably at about 50 ng/ml. HGF can be purchased from Peprotech.

The step of culturing cells with the culture medium stimulating differentiation of hepatic progenitor cells shall be carried out for the necessary time required for the production of hepatic progenitor cells. The duration of this culture step may be determined easily by one of skill in the art. For instance, during the culture the person skilled in the art can monitor the cultured cells for the absence of expression of markers only expressed by hepatic progenitor cells (e.g. Cytokeratin 19) and/or for the expression of markers specifically expressed by foetal hepatocytes (e.g. Albumin, AFP, CK18, CK8, Apolipoprotein All, Transtherythin, Alpha-1-antitrypsine, HNF4a, HNF3β, β1-integrine, c-Met, RLDL, Cyp3A7, ASGR and indocyanine green uptake and secretion). When expression of at least one, preferably several markers specific of definitive endoderm cells can not be detected and/or expression of at least one, preferably several markers specific of hepatic progenitor cells is detected, culturing with the culture medium stimulating differentiation of hepatic progenitor cells can be stopped. Monitoring of these markers can be performed using for instance RT-PCR analysis of RNA extracted from cultured cells with specific primers, immunofluorescence analysis with antibodies specific of the markers and FACS. Typically, the culture of definitive endoderm cells with said medium of the invention may be carried out for at least 3 days, preferably at least 7 days, even more preferably at least 15 days.

If necessary, the culture medium of the invention can be renewed, partly or totally, at regular intervals. Typically, the culture medium of the invention can be replaced with fresh culture medium of the invention every other day, for 15 days.

The foetal hepatocytes produced by the above method may be isolated and/or purified using any suitable method, for example FACS.

The foetal hepatocytes cells may, for example, be expanded or propagated in culture or used in clinical applications. In some embodiments, foetal hepatocytes may be further differentiated into mature hepatocytes.

The population of foetal hepatocytes of the invention may be thus suitable for obtaining mature hepatocytes.

### Pharmaceutical Compositions

The population of hepatic progenitor cells and/or foetal hepatocytes derived from definitive endoderm cells obtained according to the method of the invention may be then suitable for hepatic therapy and/or hepatic reconstruction or regeneration.

Therefore the invention relates to a pharmaceutical composition comprising a population of hepatic progenitor cells of the invention and optionally a pharmaceutically acceptable carrier or excipient. In certain embodiments, a pharmaceutical composition may further comprise at least one biologically active substance or bioactive factor.

As used herein, the term “pharmaceutically acceptable carrier or excipient” refers to a carrier medium which does not interfere with the effectiveness of the biological activity of the progenitor cells, and which is not excessively toxic to the host at the concentrations at which it is administered. Examples of suitable pharmaceutically acceptable carriers or excipients include, but are not limited to, water, salt solution (e.g., Ringer's solution), oils, gelatines, carbohydrates (e.g., lactose, amylase or starch), fatty acid esters, hydroxymethylcellulose, and polyvinyl pyrroline. Pharmaceutical compositions may be formulated as liquids, semi-liquids (e.g., gels, alginate beads) or solids (e.g., matrix, lattices, scaffolds, and the like).

As used herein the term “biologically active substance or bioactive factor” refers to any molecule or compound the presence of which in a pharmaceutical composition of the invention is beneficial to the subject receiving the composition. As will be acknowledged by one skilled in the art, biologically active substances or bioactive factors suitable for use in the practice of the present invention may be found in a wide variety of families of bioactive molecules and compounds. For example, a biologically active substance or bioactive factor useful in the context of the present invention may be selected from anti-inflammatory agents, anti-apoptotic agents, immunosuppressive or immunomodulatory agents, antioxidants, growth factors, and drugs.

A related aspect of the invention relates to a method for treating a subject suffering from a hepatic pathology, said method comprising a step of administering to the subject an efficient amount of a population of hepatic progenitor cells derived from definitive endoderm cells (or a pharmaceutical composition thereof).

According to this aspect of the invention, the hepatic pathology which may be treated is selected in the group consisting of inherited metabolic disorders (such as Crigler-Najjar Syndrome type I, glucogenosis 1a, Urea cycle defects, familial hypercholesterolemia, tyrosinemia and Wilson's Disease), chronic or acute liver failure which may be caused by viral infection (in particular infection with HBV or HCV), toxic (alcohol) and drugs, or autoimmune disorder (Autoimmune Chronic Hepatitis, Primary Biliary Cirrhosis, Primary Sclerosing Cholangitis).

As used herein, the term “efficient amount” refers to any amount of a population of hepatic progenitor cells derived from definitive endoderm cells (or a pharmaceutical composition thereof) that is sufficient to achieve the intended purpose.

The population of hepatic progenitor cells derived from definitive endoderm cells (or a pharmaceutical composition thereof) of the invention may be administered to a subject using any suitable method.

The hepatic progenitor cells derived from definitive endoderm cells of the invention may be implanted alone or in combination with other cells, and/or in combination with other biologically active factors or reagents, and/or drugs. As will be appreciated by those skilled in the art, these other cells, biologically active factors, reagents, and drugs may be administered simultaneously or sequentially with the cells of the invention.

In certain embodiments, a treatment according to the present invention further comprises pharmacologically immunosuppressing the subject prior to initiating the cell-based treatment. Methods for the systemic or local immunosuppression of a subject are well known in the art.

Effective dosages and administration regimens can be readily determined by good medical practice based on the nature of the pathology of the subject, and will depend on a number of factors including, but not limited to, the extent of the symptoms of the pathology and extent of damage or degeneration of the tissue or organ of interest, and characteristics of the subject (e.g., age, body weight, gender, general health, and the like).

### Methods for Screening Compounds

The different population of cells of the present invention may also have others uses. These uses include, but are not limited to, use for modelling injuries or pathologies associated with hepatic damage and for screening compounds in rodents.

For example said population of cells may also be used for a variety of in vitro and in vivo tests. In particular but in non limiting way, they find use in the evaluation of hepatotoxicity of compounds such as pharmaceutical candidate compounds.

Therefore, a further aspect of the invention relates to a method for screening compounds having a hepatoprotective or hepatotoxic effect wherein said method comprises the steps of:
a. culturing a population of hepatic progenitor cells, a population of foetal hepatocytes or a population of mature hepatocytes according to the invention in the presence of a test compound, and
b. comparing the survival of the cells of step a) to that of a population of said cells as defined above cultured in the absence of said test compound.

The term “hepatotoxic” refers to a compound which provokes a decrease in the survival of hepatic progenitor cells or hepatocytes. A compound is deemed to have a hepatotoxic effect if the number of viable cells cultured in the presence of said compound is lower than the number of viable cells cultured in the absence of said compound.

The term “hepatoprotective” refers to a compound which results in an increase survival of hepatic progenitor cells or neurons. A compound is deemed to have a hepatoprotective effect if the number of viable cells cultured in the presence of said compound is higher than the number of viable cells cultured in the absence of said compound. Typically, the hepatoprotective effect can be assayed in the absence of hepatotrophic factors. Alternatively, the hepatoprotective effect can be assayed in the presence of a known hepatotoxic drug. Known hepatotoxic drugs include, but are not limited to amiodarone, methotrexate, nitrofurantoin.

### Animal Model

Availability of hepatic progenitor cells and/or foetal hepatocytes which may be derived from human ES or iPS further makes it possible to design in vitro and in vivo models of human liver diseases and hepatotropic viruses, in particular hepatitis B or C. More specifically an in vivo model of human liver diseases and hepatotropic viruses may be provided by repopulating the liver of a non-human mammal with human hepatic progenitors and/or foetal hepatocytes.

Accordingly, the invention further relates to the use of human hepatic progenitor cells and/or human foetal hepatocytes obtained or obtainable by a method according to the invention for producing a non-human mammalian host which comprises functional human hepatocytes.

A suitable method to produce a chimeric non-human mammal which comprises functional human hepatocytes may comprise the step consisting of injecting into the liver of said non-human mammal human hepatic progenitor cells and/or human foetal hepatocytes according to the invention. To favour engraftment of the human hepatic progenitor cells and/or human foetal hepatocytes, the non-human mammal may receive an antimacrophage treatment to control non adaptive defense. This may be carried out for instance by administering dichloromethylene diphosphonate, e.g. by intraperitoneal injection of liposome-encapsulated dichloromethylene diphosphonate.

The invention further relates to a chimeric non-human mammal which comprises functional human hepatocytes obtained or obtainable by the method of the invention.

The non-human mammal of the invention may be any non-primate mammal into which human hepatocytes may be introduced and maintained. This includes, but is not limited to, horses, sheep, cows, cats, dogs, rats, hamsters, rabbits, gerbils, guinea pigs, and mice. Preferably, the host animal is a rodent, still preferably a mouse. It can also be non human primate (Macacus).

The non-human mammal may be in particular an immunocompromised mammal which will generally be incapable of mounting a full immune response against the xenogeneic cells (human hepatocytes). Immunocompromised mammalian hosts suitable for implantation exist or can be created, e.g., by administration of one or more compounds (e.g., cyclosporin) or due to a genetic defect which results e.g. in an inability to undergo germline DNA rearrangement at the loci encoding immunoglobulins and T-cell antigen receptors.

Functionality of the human hepatocytes can be monitored by looking at surrogate markers for hepatocyte activity, including physiologic products of human hepatocytes distinguishable from their non-human mammalian, in particular murine, analogs by immunologic or quantitative criteria, e.g., expression of human serum albumin, or expression of C-reactive protein in response to IL-6, etc. These markers can be used to determine the presence of cells without sacrifice of the recipient.

The chimeric non-human mammal which comprises functional human hepatocytes may be used in particular as an in vivo model of human hepatitis B infection.

The invention will be further illustrated by the following figures and examples. However, these examples and figures should not be interpreted in any way as limiting the scope of the present invention.

### FIGURE

FIG. 1 represents a scheme of the method according to the invention for differentiating human pluripotent or multipotent stem cells into hepatic progenitors using chemically defined medium.

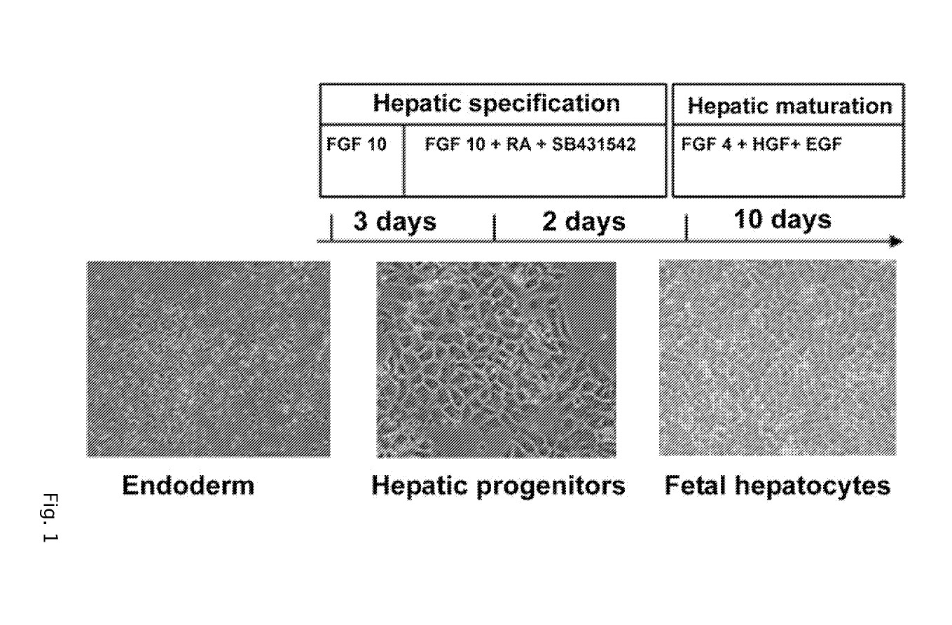

### EXAMPLE

Material & Methods

Differentiation of Definitive Endoderm (DE) Cells into Hepatic Progenitors:

To induce hepatic endoderm, DE cells were cultured in CDM-PVA during three days in presence of FGF10 (50 ng/ml, Autogenbioclear, Nottingham, UK) and then the resulting cells were grown in presence of Retinoic Acid (10⁻⁷ M, Sigma), SB431542 (10 μM, Tocris, Bristol, UK) and FGF10 (50 ng/ml, Autogenbioclear). Finally the resulting hepatic progenitors were grown in presence of FGF4 (30 ng/ml, Peprotech, Neuilly-sur-Seine, France), HGF (50 ng ml-1, Peprotech) and EGF (50 ng ml-1, Peprotech) for 3 to 15 days to drive their differentiation into hepatocytes.

RT-PCR and Quantitative PCR Analysis:

Total RNAs were extracted from cells using the RNeasy Mino Kit (Quiagen, Courtaboeuf, France). Each sample was treated with RNAse-free DNAse (Quiagen). For each sample 0.6 μg of RNA was reverse transcribed using Superscript II Reverse Transcriptase (Invitrogen). PCR amplification was performed using the GoTaq Flexi DNA Polymerase (Promega, Charbonni, France). The primers used and conditions are described in Table 1.

TABLE 1
Primers and conditions used for RT-PCR

| Gene | | | Annealing |
|---|---|---|---|
| Name | Primers Sequences | | temperature |
| Oct4 | Sense | AGTGAGAGGCAACCTGGAGA (SEQ ID NO: 1) | 60 |
| | Antisense: | ACACTCGGACCACATCCTTC (SEQ ID NO: 2) | |
| HNF4α | Sense: | CTGCTCGGAGCCACCAAGAGATCCATG (SEQ ID NO: 3) | 55 |
| | Antisense: | ATCATCTGCCACGTGATGCTCTGCA (SEQ ID NO: 4) | |
| HNF6 | Sense: | GGGCAGATGGAAGAGATCAA (SEQ ID NO: 5) | 55 |
| | Antisense: | TGCGTTCATGAAGAAGTTGC (SEQ ID NO: 6) | |
| CEBPα | Sense: | CTCGAGGCTTGCCCAGACCGT (SEQ ID NO: 7) | 58 |
| | Antisense: | GCGGGCTTGTCGGGATCTCAG (SEQ ID NO: 8) | |
| AFP | Sense: | AGAACCTGTCACAAGCTGTG (SEQ ID NO: 9) | 58 |
| | Antisense: | GACAGCAAGCTGAGGATGTC (SEQ ID NO: 10) | |
| ALB | Sense: | CCTTTGGCACAATGAAGTGGGTAACC (SEQ ID NO: 11) | 55 |
| | Antisense: | CAGCAGTCAGCCATTCACCATAGG (SEQ ID NO: 12) | |
| AAT | Sense: | AGACCCTTTGAAGTCAAGCGACC (SEQ ID NO: 13) | 55 |
| | Antisense: | CCATTGCTGAAGACCTTAGTGATGC (SEQ ID NO: 14) | |
| TO | Sense: | GGCAGCGAAGAAGTACAAATC (SEQ ID NO: 15) | 55 |
| | Antisense: | TCGAACAGAATCCAACTCCC (SEQ ID NO: 16) | |
| TAT | Sense: | TACAGACCCTGAAGTTACCCAG (SEQ ID NO: 17) | 55 |
| | Antisense: | TAAGAAGCAATCTCCTCCCGA (SEQ ID NO: 18) | |
| ApoAll | Sense: | GGAGAAGGTCAAGAGCCCGAG (SEQ ID NO: 19) | 60 |
| | Antisense: | AGCAAAGAGTGGGTAGGGACAG (SEQ ID NO: 20) | |
| Facteur | Sense: | TGTTGGTGTCCCTTTGGATT (SEQ ID NO: 21) | 55 |
| IX | Antisense: | TCACTCAAAGCACCCAATCA (SEQ ID NO: 22) | |
| BilUGT | Sense: | ATGACCCGTGCCTTTATCAC (SEQ ID NO: 23) | 60 |
| | Antisense: | TCTTGGATTTGTGGGCTTTC (SEQ ID NO: 24) | |
| Cyp7A1 | Sense: | AGAAGGCAAACGGGTGAACC (SEQ ID NO: 25) | 60 |
| | Antisense: | GGGTCAATGCTTCTGTGCCC (SEQ ID NO: 26) | |
| β2m | Sense: | ACTGAAAAAGATGAGTATGCCTGCCGTGTGAACC (SEQ ID NO: 27) | 55 |
| | Antisense: | CCTGCTCAGATACATCAAACATGGAGACAGCACT (SEQ ID NO: 28) | |

Real Time RT-PCR was performed using a Stratagen Mw3005P and the mixture was prepared as described by the manufacturer (SensiMiX Protocol Quantace, London, UK) then denatured at 94° C. for 30 seconds, 60° C. for 30 seconds, and 72° C. for 30 seconds followed by final extension at 72° C. for 10 minutes after completion of 40 cycles.

The primers used for the Quantitative PCR are described in Table 2. Each reaction was performed in duplicate and normalized to PBGD on the same run. The results are presented as the mean of three independent experiments and error bars indicate standard deviation.

TABLE 2
Primers used for RT-QPCR

| Gene Name | Primers sequences | |
|---|---|---|
| oct-04 | Sense: | AGT GAG AGG CAA CCT GGA GA (SEQ ID NO: 29) |
| | Antisense: | ACA CTC GGA CCA CAT CCT TC (SEQ ID NO: 30) |
| Sox17 | Sense: | GAT ACG CCA GTG ACG ACC AGA (SEQ ID NO: 31) |
| | Antisense: | ATC TTG CTC AAC TCG GCG TT (SEQ ID NO: 32) |
| HNF3b | Sense: | GGG AGC GGT GAA GAT GGA (SEQ ID NO: 33) |
| | Antisense: | TCA TGT TGC TCA CGG AGG AGT A (SEQ ID NO: 34) |
| HNF1b | Sense: | TCA CAG ATA CCA GCA GCA TCA GT (SEQ ID NO: 35) |
| | Antisense: | GGG CAT CAC CAG GCT TGT A (SEQ ID NO: 36) |
| HNF4 | Sense: | CAT GGC CAA GAT TGA AAC CT (SEQ ID NO: 37) |
| | Antisense: | TTC CCA TAT GTT CCT GCA TCA G (SEQ ID NO: 38) |
| HNF6 | Sense: | CGC TCC GCT TAG CAG CAT (SEQ ID NO: 39) |
| | Antisense: | GTG TTG CCT CTA TCC TTC CCA T (SEQ ID NO: 40) |
| Wnt3 | Sense: | CTC GCT GGC TAC CCA ATT (SEQ ID NO: 41) |
| | Antisense: | AGA AGC GCA GTT GCT TGG (SEQ ID NO: 42) |
| AFP | Sense: | ACCATGAAGTGGGTGGAATC (SEQ ID NO: 43) |
| | Antisense: | TGGTAGCCAGGTCAGCTAAA (SEQ ID NO: 44) |
| Alb | Sense: | TTGGCACAATGAAGTGGGTA (SEQ ID NO: 45) |
| | Antisense: | AAAGGCAATCAACACCAAGG (SEQ ID NO: 46) |
| Sox7 | Sense: | CAT GCA GGA CTA CCC CAA CT (SEQ ID NO: 47) |
| | Antisense: | GCT ACA GTG GAG AGG GCT TG (SEQ ID NO: 48) |
| hHex | Sense: | GCGAGAGACAGGTCAAAACC (SEQ ID NO: 49) |
| | Antisense: | AGGGCGAACATTGAGAGCTA (SEQ ID NO: 50) |
| E-Cadherin | Sense: | GCT GGA GAT TAA TCC GGA CA (SEQ ID NO: 51) |
| | Antisense: | ACC TGA GGC TTT GGA TTC CT (SEQ ID NO: 52) |
| MixL1 | Sense: | CCG AGT CCA GGA TCC AGG TA (SEQ ID NO: 53) |
| | Antisense: | CTC TGA CGC CGA GAC TTG G (SEQ ID NO: 54) |

Immunofluorescence:

Cells were fixed for 20 minutes at 4° C. in paraformaldehyde 4% (Alpha Aesar, Karlsruhe, Germany) then blocked one hour in a PBS solution containing 3% BSA or 1% gelatin. For intracellular staining cells were permeabilized in 0.1% Triton X-100 before blocking. The cells were incubated one hour at room temperature with the primary antibodies. Primary antibodies against human alpha-1-antitrypsin (1:100), CK19 (1:50), and α-fetoprotein (1:300) were purchased from DAKO (DakoCytomation, Trappes, France) Antibodies against human Oct4 (1:100), HNF4 (1:100) were purchased from Tebu Bio (Le Perray en Yvelines, France). After three washes in PBS cells, cells were incubated one hour at room temperature with secondary antibodies, goat anti-mouse Cy3 (1:800) conjugated and chicken anti-rabbit alexa 488 (1:600) conjugated were obtained from GE-HealthCare Bio-Sciences AB. Unbound secondary antibodies were removed by 3 washes in PBS. Hoescht 33258 (1:10000, Sigma) was added to the first wash.

FACS Analysis (Flow Cytometry):

Cells were harvested by dissociation for 5 min at 37° C. with 0.2 mg/ml EDTA (Sigma) and 1 mg/ml BSA fraction V (Sigma) in PBS washed and resuspended in PBS+3% FBS. Cells were incubated at 4° C. with primaries antibodies rabbit anti-human c-met (1:25) (Tebu Bio), or rabbit anti-human ASGr (1:25) (Abcam Cambridge, UK), rabbit anti-human rLDL (1:20) (Abcam) or a CD-49f FITC-conjugated antibody (1:20) (BD Pharmingen, Brumath, France). After 3 washes, cells were incubated with an antibody PE-conjugated goat anti rabbit (1:100). Cells were then analysed using a FACS-Calibur (BD Biosciences).

Hepatocyte Functions

Glycogen storage was assayed by the Periodate-Schiff technique according to McManus.

Uptake of LDL was performed using Dil-Ac-LDL staining kit (Biomedical Technologies, Stoughton, Mass.) and the assay was performed according to the manufacturer's instructions. For co-localization with hepatic markers, cells were fixed in 4% paraformaldehyde and then further assayed by immunofluorescence as described above.

Albumin concentrations were measured with a kit specific for human protein (Dade Behring).

The Indocyanine green (ICG) uptake test was performed by incubation of the cells with 1 mg/ml ICG for 60 min. Cells were then washed in medium and release of ICG was evaluated 16 hours later.

CYP3A7 activity was measured using the P450-Glo assays kit (Promega) according manufacturer recommendation. Cytochrome activity was then analysed using P450-GloMax 96 microplate luminometer.

Lentivirus Production and Transduction of Human Embryonic Stem Cells

The EF1α-GFP lentivector was constructed and produced by Vectalys (Toulouse, France). The APOA-II-GFP lentivector was constructed in the laboratory and produced by Vectalys.

Prior transduction with lentiviruses, hESCs were dissociated and were incubated with viral particles for 3 hours at 37° C. in low-attachment 24-well plate (Corning Life Sciences) under gentle rocking before seeding onto mitotically inactivated MEF in hESC medium containing 4 ng/ml FGF2 (R&D systems). Undifferentiated transduced cells were expanded and differentiated using chemically defined conditions described above.

Animals:

Animal studies were conducted under protocols approved by the French Ministry of Agriculture. Differentiated cells (5×10⁵ cells/animal in 50 μl Saline solution) were injected into the liver of five-day-old uPAxRag2gammac^{-/-} mice (n=9). To control non adaptive defense, mice received antimacrophage treatment by intraperitoneal injection of liposome-encapsulated dichloromethylene diphosphonate (250 μg of clodronate) as described in (Strick-Marchand et al., 2004). The transplanted mice were killed eight weeks after transplantation. Blood samples were collected and human albumin was quantified in sera by Elisa Test. Livers were removed for histologic analysis and liver fragments were either embedded in OCT compounds then frozen in liquid nitrogen, or were fixed in PFA 4% and embedded in paraffin. Non-transplanted mice were used as controls.

Results

Differentiation of Human Endoderm Cells into Hepatic Progenitors

DE cells were generated by culturing H9 cells or hIPSCs for 2 days in CDM+10 ng/ml Activin+FGF2 12 ng/ml, and for 3 days in CDM-PVA, 100 ng/ml Activin, 20 ng/ml FGF2, 10 ng/ml BMP4, 10 μM LY294002. The capacity of DE cells to differentiate further into liver progenitors in the presence of diverse combinations of factors such as FGF10, Wnt, Retinoic Acid (RA), and Activin A. The effect of inhibiting the Activin and Wnt pathways was also analysed using respectively SB431542, a pharmacological inhibitor of Activin/Nodal receptors (Inman et al., 2002), or Frizzled8/Fc chimera an inhibitor of Wnt signalling pathway (Hsieh et al., 1999).

Generation of liver cells was monitored for the expression of HNF4alpha and alpha-Fetoprotein (AFP), two markers expressed in hepatic progenitors during the early stages of liver development. The highest induction in HNF4a and AFP expression was observed when DE cells were first grown for 3 days in the presence of FGF10, and then for 2 additional days in a combination of FGF10, RA and SB431542.

Inhibition of Wnt signalling decreased hepatic differentiation, which confirms recent observations showing that Wnt has an essential function in the mechanisms controlling the differentiation of DE cells into hepatic endoderm (Hay et al., 2008). However, exogenous Wnt was not necessary in our culture conditions since DE cells already expressed a high level of Wnt3a.

These results suggest that RA plays an important role in hepatic specification, which is synergised by FGF10 and Wnt signalling, whereas TGFβ signalling might interfere with the early step of DE cells differentiation into liver progenitors.

Most of the hepatic progenitors generated by the combination of FGF10, RA and SB431542 expressed EpCAM. HNF4a and AFP or CK19 were co-expressed in 60% and 50% of the cells respectively. Since during liver development hepatocytes and biliary epithelial cells derive from a common bipotential progenitor (hepatoblasts), our results suggest that a population of hepatoblasts has been generated during the differentiation process.

Maturation of Hepatic Progenitors into Hepatocyte-Like Cells

Culture conditions were then developed to differentiate hepatic progenitors into hepatocytes. We observed that combination of FGF4, HGF and EGF was sufficient to drive differentiation of hepatic progenitors into more differentiated cells. After 5 days in these culture conditions, the morphology of the cells resembled the cuboidal shapes typical of hepatocytes. In addition, the cells generated expressed markers specific for mature liver cells including alpha1-anti-trypsin (AAT), Apolipoprotein A-II (ApoAII), Tyrosine Aminotransferase, tryptophan 2,3-dioxygenase, Factor IX and the detoxifying enzymes Cyp3A7 and Cyp7A1 as compared with human fetal and adult hepatocytes.

Generation of hepatocytes was confirmed by immunostaining analyses showing that differentiating hepatic progenitors expressed near homogeneously CK8/18 and that clusters of cells expressed AAT and high level of Albumin (Alb). FACS analyses showed that 35% of the cells expressed ASGR1, LDLR, c-met and alpha6 integrin. Interestingly, these two last cell surface markers are hallmarks of proliferating hepatic cells in vivo.

To further confirm the identity of these hepatocyte-like cells, we transduced undifferentiated hESC with a recombinant lentivirus carrying the GFP under the control of the human APOA-II regulatory sequences and induced their differentiation. Cells transduced with a lentivector carrying the GFP under the control of EF1alpha promoter were used as positive control. Flow cytometry analyses showed that 75% of the control cells were fluorescent 48 hours after transduction whereas expression of GFP was not detectable when driven by APOA-II promoter. Cells transduced with APOA-II-GFP lentivector started to express GFP only after 13 days of differentiation confirming the progressive differentiation of endoderm cells into mature liver cells and also demonstrating that hepatic cells generated in our culture system display the physiological regulation of a hepatic-specific promoter.

In addition, we tested whether these differentiated cells were functional in vitro. Periodic Acid Schiff staining revealed that 60% of the cells could store glycogen. In addition CK19-positive cells and AFP-positive cells were capable to uptake LDL. We also examined uptake and excretion of ICG indocyanin green, which is a functional characteristic of hepatocytes. ICG-positive cells were visible and the cells had excluded ICG by 16 h after its removal from the medium. Analysis of the secreted amount of albumin in the culture medium revealed that these cells secreted albumin at a rate of 5.9+0.7 μg/10⁶ cells/day. Finally, generated hepatocytes displayed CYP3A7 activity.

ES-Derived Hepatic Cells are Functional In Vivo

It was then investigated the capacity of hepatocytes generated from hESCs to engraft and to differentiate within liver parenchyma. GFP-expressing hESCs were differentiated for 21 days and the resulting cells were transplanted into the liver uPAxrag2 gammac^{-/-} mice. These immunodeficient transgenic mice express the urokinase gene under the control of Alb promoter. This transgene is toxic for hepatocytes and thus it blocks transiently liver growth (until transgene is inactivated in resident cells), allowing a better engraftment of the transplanted cells.

Immunohistochemical analyses showed the presence of cells expressing human AAT and ALB in the liver of transplanted mice confirming that hepatic cells generated from hESCs were capable to engraft in vivo and to express proteins characteristics of hepatocytes. Human cells were distributed throughout the liver mainly as small and large cell clusters, suggesting that transplanted cells had proliferated and participated in liver growth. In addition, human AAT and GFP protein were co-expressed in the same cells confirming the human origin of these cell clusters. Furthermore, the serum of transplanted animals contained 3 ng/ml human albumin confirming that the transplanted cells displayed in vivo some functions characteristics of hepatocytes. Finally, histological examination did not reveal the presence of teratomas or intra hepatic tumor suggesting that the cell population injected only contain fully differentiated cells.

Generation of Hepatic Progenitors from Human Induced Pluripotent Stem Cells Using Culture Conditions Developed for hESCs

Human induced pluripotent stem cells can be derived from reprogrammed fibroblasts. Therefore, we investigated whether the culture conditions developed to generate hepatocytes from hESCs could also be efficient in differentiating hIPSCs into hepatic cells. Foreskin fibroblast were reprogrammed in CDM+Activin A+FGF2 as described (Vallier et al., 2009) using retrovirus expressing Oct-4, Sox2, KLF4, and cMyc and three of the resulting hIPSCs lines were grown in the culture conditions described above. Immunostaining and Q-PCR analyses showed that the cells generated under these culture conditions expressed HNF4a, AFP, and Albumin at similar level than hESCs differentiated using the same culture conditions. All together, these data suggest that our approach developed with hESCs can be used to generate liver cells from hIPSCs.

While several methods are currently available to generate hepatocyte-like cells from hESCs, our approach has two major advantages. It is based on fully defined media devoid of feeder cells and serum and it also avoids the use of Sodium Butyrate or DMSO both of which are known to affect the epigenetic profile of mammalian cells by respectively inhibiting histone acetylation and increasing DNA methylation (Iwatani et al., 2006). Consequently, our method brings clear improvement on existing methods allowing the differentiation of hESCs into hepatocytes (Cai et al., 2007; Duan et al., 2007; Lavon et al., 2004; Rambhatla et al., 2003; Schwartz et al., 2005).

Furthermore, our protocol follows a process that mimics the progressive differentiation of definitive endoderm cells into hepatic cells during mammalian development. In the first step, hESCs are differentiated into DE cells using combination of high dose of Activin A, FGF2, BMP4 and Ly294002. The use of this PI3 kinase inhibitor to increase endoderm differentiation of hESCs has been shown previously (Johansson and Wiles, 1995). However, this study was based on media containing serum, matrigel and feeders. In addition, inhibition of PI3 kinase in our conditions was not sufficient to block the effect of Activin signalling on pluripotency. Consequently, other factors such as BMP4 are required to repress the expression of pluripotent genes and to transform Activin signalling into an inductive signal for endoderm differentiation. These results emphasize the importance to use fully defined culture conditions to define signalling pathways controlling key cell fate choice. In the second step, combination of FGF10 and RA, two factors known to be critical for liver growth and for hepatoblasts survival in vivo (Hatzis and Talianidis, 2001; Berg et al, 2007; Zaret, 2008) appear to be essential to drive differentiation of endoderm cells into hepatic progenitors co-expressing CK19 and HNF4a. These two markers are specifically co-expressed by bipotential hepatoblasts at early stage of development and their expression becomes segregated between biliary epithelial cells and hepatocytes respectively during liver organogenesis. However, we also observed that inhibition of Activin signalling can increase hepatic differentiation, by contrast to studies in the mouse embryo showing that TGFβ is necessary for liver bud formation (Lemaigre and Zaret, 2004). However, the function of Activin/TGFβ signalling in mouse and human liver development might not be conserved. Alternatively, liver bud specification takes 3-4 days in human against 12-24 hours in the mouse and thus, mechanisms happening very quickly during mouse development could become more evident during differentiation of hESCs in vitro.

The third step consists in differentiating these hepatic progenitors into hepatocytes while maintaining a proliferative status using a combination of growth factors (FGF4, EGF and HGF) known to be involved in this process in vivo (Jung et al., 1999; Suzuki et al., 2003). The cells generated expressed various adult liver-specific proteins, as well as key hepatocyte nuclear factors required for controlling the expression of many liver-specific genes. In addition these cells also exhibited specific function of liver cells. Although these characteristics suggest that hESCs-derived hepatocytes are differentiated, our results also suggest that this population is still at fetal liver developmental stage. Indeed, these hepatocytes retain some immature characteristics such as expression of AFP as already reported by others (Basma et al., 2009; Cai et al., 2007). Further investigations will be required to determine conditions for generating fully mature hepatocytes. Additional inducing factors in combination with high-cell-density culture or co-culture with other cell types such as endothelial cells might represent a potential solution to overcome this major challenge.

Our study also demonstrates that hESCs-derived hepatocytes engrafted efficiently within host liver parenchyma while retaining the ability to proliferate and to display characteristics of normal differentiated hepatocytes. However, low amount of human albumin in serum of transplanted mouse suggests a lack of correlation between human protein secretion and engraftment efficacy. This has been already reported by one of us and others in this model (Mahieu-Caputo et al., 2004). One explanation is that the degree of hepatic cell differentiation is partial 2 months after transplantation and that engrafted cells produce less than expected amount of albumin. The xenogenic environment could also underestimate the capacity of human progenitors to fully differentiate.

Tumor formation and abnormal growth remain another major issue when using hES-derived cells in vivo. Indeed, generated populations can be easily contaminated by undifferentiated pluripotent cells which have the capacity to form teratomas (D'Amour et al., 2006; Kroon et al., 2008). In addition, adult environment might not be capable to control the proliferative capacity of early progenitors leading to uncontrolled proliferation. Thus, it was recently reported that hESC-derived AFP-producing cells induced teratomas formation (Ishii et al., 2007). Adenocarcinomas were also observed intraperitoneally in analbuminemic rats transplanted with hESC-derived hepatocytes (Basma et al., 2009). Importantly, hepatic cells generated using our three-step approach did not produce tumors after transplantation, suggesting that our method induces differentiation of the totality of pluripotent cells.

While the capacity to generate large quantity of hepatocytes presents obvious advantages for in vitro studies such as pharmacotoxicology, the utilisation of terminally differentiated cells might present several drawbacks for cell-based therapy. Indeed, numerous studies in rodents have demonstrated that hepatocytes do not proliferate in vivo unless they display a proliferative advantage (Azuma et al., 2007). The use of progenitors/fetal hepatocytes could represent an advantageous alternative, which might solve these major limitations. Indeed we previously isolated such immature cells from early developmental stages of human liver and shown that they were able to differentiate and to proliferate in rodent livers (Mahieu-Caputo et al., 2004). Moreover, the proof of principle that progenitors can be used efficiently for cell-based therapy has been clearly established for other organ such as neuronal stem cells in the context of neurodegenerative diseases (Bachoud-Levi et al., 2006).

In any case, mouse models will not allow by themselves to determine whether differentiated cells will be safe for clinical applications. Indeed, the quantity of cells transplanted is limited by the size of the organs and studies on large animals such as nonhuman primates will be needed to carefully address safety issues.

In conclusion, we have provided the first demonstration that fully defined conditions mimicking liver development results in generation of functional hepatocytes in vitro and in vivo. We also showed that this approach is directly transposable to pluripotent stem cells generated by reprogramming of adult somatic cells from individual patient. This study opens up the way for the production of liver cells from hESCs or hiPSCs reprogrammed from patients suffering of liver diseases. This should allow the creation of new in vitro models to perform drug screening and to develop new therapies for liver diseases.

### REFERENCES

Throughout this application, various references describe the state of the art to which this invention pertains. The disclosures of these references are hereby incorporated by reference into the present disclosure.
Aurich, H., Sgodda, M., Kaltwasser, P., Vetter, M., Weise, A., Liehr, T., Brulport, M., Hengstler, J. G., Dollinger, M. M., Fleig, W. E., et al. (2009). Hepatocyte differentiation of mesenchymal stem cells from human adipose tissue in vitro promotes hepatic integration in vivo. Gut 58, 570-581.
Azuma, H., Paulk, N., Ranade, A., Dorrell, C., Al-Dhalimy, M., Ellis, E., Strom, S., Kay, M. A., Finegold, M., and Grompe, M. (2007). Robust expansion of human hepatocytes in Fah−/−/Rag2−/−/II2rg−/− mice. Nat Biotechnol 25, 903-910.
Bachoud-Levi, A. C., Gaura, V., Brugieres, P., Lefaucheur, J. P., Boisse, M. F., Maison, P., Baudic, S., Ribeiro, M. J., Bourdet, C., Remy, P., et al. (2006). Effect of fetal neural transplants in patients with Huntington's disease 6 years after surgery: a long-term follow-up study. Lancet Neurol 5, 303-309.
Basma, H., Soto-Gutierrez, A., Yannam, G. R., Liu, L., Ito, R., Yamamoto, T., Ellis, E., Carson, S. D., Sato, S., Chen, Y., et al. (2009). Differentiation and transplantation of human embryonic stem cell-derived hepatocytes. Gastroenterology 136, 990-999.
Berg T, Rountree C B, Lee L, Estrada J, Sala F G, Choe A, Veltmaat J M, De Langhe S, Lee R, Tsukamoto H, Crooks G M, Bellusci S, Wang K S (2007). Fibroblast growth factor 10 is critical for liver growth during embryogenesis and controls hepatoblast survival via beta-catenin activation. Hepatology 46, 1187-1197.
Cai, J., Zhao, Y., Liu, Y., Ye, F., Song, Z., Qin, H., Meng, S., Chen, Y., Zhou, R., Song, X., et al. (2007). Directed differentiation of human embryonic stem cells into functional hepatic cells. Hepatology 45, 1229-1239.
Chiao, E., Elazar, M., Xing, Y., Xiong, A., Kmet, M., Millan, M. T., Glenn, J. S., Wong, W. H., and Baker, J. (2008). Isolation and transcriptional profiling of purified hepatic cells derived from human embryonic stem cells. Stem Cells 26, 2032-2041.
Chung, Y., Klimanskaya, I., Becker, S., Li, T., Maserati, M., Lu, S. J., Zdravkovic, T., Ilic, D., Genbacev, O., Fisher, S., et al. (2008). Human embryonic stem cell lines generated without embryo destruction. Cell Stem Cell 2, 113-117.
Crettaz, M., Baron, A., Siegenthaler, G., and Hunziker, W. (1990). Ligand specificities of recombinant retinoic acid receptors RAR alpha and RAR beta. Biochem J 272, 391-397.
D'Amour, K. A., Bang, A. G., Eliazer, S., Kelly, O. G., Agulnick, A. D., Smart, N. G., Moorman, M. A., Kroon, E., Carpenter, M. K., and Baetge, E. E. (2006). Production of pancreatic hormone-expressing endocrine cells from human embryonic stem cells. Nat Biotechnol 24, 1392-1401.
D'Ippolito, G., Diabira, S., Howard, G. A., Menei, P., Roos, B. A., and Schiller, P. C. (2004). Marrow-isolated adult multilineage inducible (MIAMI) cells, a unique population of postnatal young and old human cells with extensive expansion and differentiation potential. J Cell Sci 117, 2971-2981.
Dellavalle, A., Sampaolesi, M., Tonlorenzi, R., Tagliafico, E., Sacchetti, B., Perani, L., Innocenzi, A., Galvez, B. G., Messina, G., Morosetti, R., et al. (2007). Pericytes of human skeletal muscle are myogenic precursors distinct from satellite cells. Nat Cell Biol 9, 255-267.
Duan, Y., Catana, A., Meng, Y., Yamamoto, N., He, S., Gupta, S., Gambhir, S. S., and Zern, M. A. (2007). Differentiation and enrichment of hepatocyte-like cells from human embryonic stem cells in vitro and in vivo. Stem Cells 25, 3058-3068.
Fox, I. J., and Roy-Chowdhury, J. (2004). Hepatocyte transplantation. J Hepatol 40, 878-886.
Grenman, R., Carey, T. E., McClatchey, K. D., Wagner, J. G., Pekkola-Heino, K., Schwartz, D. R., Wolf, G. T., Lacivita, L. P., Ho, L., Baker, S. R., et al. (1991). In vitro radiation resistance among cell lines established from patients with squamous cell carcinoma of the head and neck. Cancer 67, 2741-2747.
Hatzis, P., and Talianidis, I. (2001). Regulatory mechanisms controlling human hepatocyte nuclear factor 4alpha gene expression. Mol Cell Biol 21, 7320-7330.
Hay, D. C., Fletcher, J., Payne, C., Terrace, J. D., Gallagher, R. C., Snoeys, J., Black, J. R., Wojtacha, D., Samuel, K., Hannoun, Z., et al. (2008). Highly efficient differentiation of hESCs to functional hepatic endoderm requires ActivinA and Wnt3a signaling. Proc Natl Acad Sci USA 105, 12301-12306.
Horn, V., Minucci, S., Ogryzko, V. V., Adamson, E. D., Howard, B. H., Levin, A. A., and Ozato, K. (1996). RAR and RXR selective ligands cooperatively induce apoptosis and neuronal differentiation in P19 embryonal carcinoma cells. FASEB J 10, 1071-1077.
Hsieh, J. C., Rattner, A., Smallwood, P. M., and Nathans, J. (1999). Biochemical characterization of Wnt-frizzled interactions using a soluble, biologically active vertebrate Wnt protein. Proc Natl Acad Sci USA 96, 3546-3551.
Inman, G. J., Nicolas, F. J., Callahan, J. F., Harling, J. D., Gaster, L. M., Reith, A. D., Laping, N. J., and Hill, C. S. (2002). SB-431542 is a potent and specific inhibitor of transforming growth factor-beta superfamily type I activin receptor-like kinase (ALK) receptors ALK4, ALK5, and ALK7. Mol Pharmacol 62, 65-74.
Ishii, T., Yasuchika, K., Machimoto, T., Kamo, N., Komori, J., Konishi, S., Suemori, H., Nakatsuji, N., Saito, M., Kohno, K., et al. (2007). Transplantation of embryonic stem cell-derived endodermal cells into mice with induced lethal liver damage. Stem Cells 25, 3252-3260.
Iwatani, M., Ikegami, K., Kremenska, Y., Hattori, N., Tanaka, S., Yagi, S., and Shiota, K. (2006). Dimethyl sulfoxide has an impact on epigenetic profile in mouse embryoid body. Stem Cells 24, 2549-2556.
Jiang, Y., Jahagirdar, B. N., Reinhardt, R. L., Schwartz, R. E., Keene, C. D., Ortiz-Gonzalez, X. R., Reyes, M., Lenvik, T., Lund, T., Blackstad, M., et al. (2002). Pluripotency of mesenchymal stem cells derived from adult marrow. Nature 418, 41-49.
Johansson, B. M., and Wiles, M. V. (1995). Evidence for involvement of activin A and bone morphogenetic protein 4 in mammalian mesoderm and hematopoietic development. Mol Cell Biol 15, 141-151.
Jung, J., Zheng, M., Goldfarb, M., and Zaret, K. S. (1999). Initiation of mammalian liver development from endoderm by fibroblast growth factors. Science 284, 1998-2003.
Kogler, G., Sensken, S., Airey, J. A., Trapp, T., Muschen, M., Feldhahn, N., Liedtke, S., Sorg, R. V., Fischer, J., Rosenbaum, C., et al. (2004). A new human somatic stem cell from placental cord blood with intrinsic pluripotent differentiation potential. J Exp Med 200, 123-135.
Krause, C. J., Carey, T. E., Ott, R. W., Hurbis, C., McClatchey, K. D., and Regezi, J. A. (1981). Human squamous cell carcinoma. Establishment and characterization of new permanent cell lines. Arch Otolaryngol 107, 703-710.
Kroon, E., Martinson, L. A., Kadoya, K., Bang, A. G., Kelly, O. G., Eliazer, S., Young, H., Richardson, M., Smart, N. G., Cunningham, J., et al. (2008). Pancreatic endoderm derived from human embryonic stem cells generates glucose-responsive insulin-secreting cells in vivo. Nat Biotechnol 26, 443-452.
Lavon, N., Yanuka, O., and Benvenisty, N. (2004). Differentiation and isolation of hepatic-like cells from human embryonic stem cells. Differentiation 72, 230-238.
Lemaigre, F., and Zaret, K. S. (2004). Liver development update: new embryo models, cell lineage control, and morphogenesis. Curr Opin Genet Dev 14, 582-590.
Mahieu-Caputo, D., Allain, J. E., Branger, J., Coulomb, A., Delgado, J. P., Andreoletti, M., Mainot, S., Frydman, R., Leboulch, P., Di Santo, J. P., et al. (2004). Repopulation of athymic mouse liver by cryopreserved early human fetal hepatoblasts. Hum Gene Ther 15, 1219-1228.
Michieli, P., Cavassa, S., Basilico, C., De Luca, A., Mazzone, M., Asti, C., Chiusaroli, R., Guglielmi, M., Bossu, P., Colotta, F., et al. (2002). An HGF-MSP chimera disassociates the trophic properties of scatter factors from their pro-invasive activity. Nat Biotechnol 20, 488-495.
Puppi, J., and Dhawan, A. (2009). Human hepatocyte transplantation overview. Methods Mol Biol 481, 1-16.
Rambhatla, L., Chiu, C. P., Kundu, P., Peng, Y., and Carpenter, M. K. (2003). Generation of hepatocyte-like cells from human embryonic stem cells. Cell Transplant 12, 1-11.
Reiss, M., Pitman, S. W., and Sartorelli, A. C. (1985). Modulation of the terminal differentiation of human squamous carcinoma cells in vitro by all-trans-retinoic acid. J Natl Cancer Inst 74, 1015-1023.
Revazova, E. S., Turovets, N. A., Kochetkova, O. D., Kindarova, L. B., Kuzmichev, L. N., Janus, J. D., and Pryzhkova, M. V. (2007). Patient-specific stem cell lines derived from human parthenogenetic blastocysts. Cloning Stem Cells 9, 432-449.
Reyes, M., Dudek, A., Jahagirdar, B., Koodie, L., Marker, P. H., and Verfaillie, C. M. (2002). Origin of endothelial progenitors in human postnatal bone marrow. J Clin Invest 109, 337-346.
Rupniak, H. T., Rowlatt, C., Lane, E. B., Steele, J. G., Trejdosiewicz, L. K., Laskiewicz, B., Povey, S., and Hill, B. T. (1985). Characteristics of four new human cell lines derived from squamous cell carcinomas of the head and neck. J Natl Cancer Inst 75, 621-635.
Sacks, P. G., Oke, V., Amos, B., Vasey, T., and Lotan, R. (1989). Modulation of growth, differentiation and glycoprotein synthesis by beta-all-trans retinoic acid in a multicellular tumor spheroid model for squamous carcinoma of the head and neck. Int J Cancer 44, 926-933.
Sacks, P. G., Parnes, S. M., Gallick, G. E., Mansouri, Z., Lichtner, R., Satya-Prakash, K. L., Pathak, S., and Parsons, D. F. (1988). Establishment and characterization of two new squamous cell carcinoma cell lines derived from tumors of the head and neck. Cancer Res 48, 2858-2866.
Sampaolesi, M., Blot, S., D'Antona, G., Granger, N., Tonlorenzi, R., Innocenzi, A., Mognol, P., Thibaud, J. L., Galvez, B. G., Barthelemy, I., et al. (2006). Mesoangioblast stem cells ameliorate muscle function in dystrophic dogs. Nature 444, 574-579.
Schwartz, R. E., Linehan, J. L., Painschab, M. S., Hu, W. S., Verfaillie, C. M., and Kaufman, D. S. (2005). Defined conditions for development of functional hepatic cells from human embryonic stem cells. Stem Cells Dev 14, 643-655.
Shi, Y., and Massague, J. (2003). Mechanisms of TGF-beta signaling from cell membrane to the nucleus. Cell 113, 685-700.
Shirahashi, H., Wu, J., Yamamoto, N., Catana, A., Wege, H., Wager, B., Okita, K., and Zern, M. A. (2004). Differentiation of human and mouse embryonic stem cells along a hepatocyte lineage. Cell Transplant 13, 197-211.
Smith J R, Vallier L, Lupo G, Alexander M, Harris W A, Pedersen R A. (2008). Inhibition of Activin/Nodal signaling promotes specification of human embryonic stem cells into neuroectoderm. Dev Biol. 313(1), 107-17.
Strick-Marchand, H., Morosan, S., Charneau, P., Kremsdorf, D., and Weiss, M. C. (2004). Bipotential mouse embryonic liver stem cell lines contribute to liver regeneration and differentiate as bile ducts and hepatocytes. Proc Natl Acad Sci USA 101, 8360-8365.
Sun, S. Y., Yue, P., Dawson, M. I., Shroot, B., Michel, S., Lamph, W. W., Heyman, R. A., Teng, M., Chandraratna, R. A., Shudo, K., et al. (1997). Differential effects of synthetic nuclear retinoid receptor-selective retinoids on the growth of human non-small cell lung carcinoma cells. Cancer Res 57, 4931-4939.
Sun, S. Y., Yue, P., Mao, L., Dawson, M. I., Shroot, B., Lamph, W. W., Heyman, R. A., Chandraratna, R. A., Shudo, K., Hong, W. K., et al. (2000). Identification of receptor-selective retinoids that are potent inhibitors of the growth of human head and neck squamous cell carcinoma cells. Clin Cancer Res 6, 1563-1573.
Suzuki, A., Iwama, A., Miyashita, H., Nakauchi, H., and Taniguchi, H. (2003). Role for growth factors and extracellular matrix in controlling differentiation of prospectively isolated hepatic stem cells. Development 130, 2513-2524.
Takahashi, K., Tanabe, K., Ohnuki, M., Narita, M., Ichisaka, T., Tomoda, K., and Yamanaka, S. (2007). Induction of pluripotent stem cells from adult human fibroblasts by defined factors. Cell 131, 861-872.
Vallier, L., Touboul, T., Chng, Z., Brimpari, M., Hannan, N., Millan, E., Smithers, L. E., Trotter, M., Rugg-Gunn, P., Weber, A., et al. (2009). Early cell fate decisions of human embryonic stem cells and mouse epiblast stem cells are controlled by the same signalling pathways. PLoS ONE 4, e6082.
Yeh, B. K., Igarashi, M., Eliseenkova, A. V., Plotnikov, A. N., Sher, I., Ron, D., Aaronson, S. A., and Mohammadi, M. (2003). Structural basis by which alternative splicing confers specificity in fibroblast growth factor receptors. Proc Natl Acad Sci USA 100, 2266-2271.
Yu, J., Vodyanik, M. A., Smuga-Otto, K., Antosiewicz-Bourget, J., Frane, J. L., Tian, S., Nie, J., Jonsdottir, G. A., Ruotti, V., Stewart, R., et al. (2007). Induced pluripotent stem cell lines derived from human somatic cells. Science 318, 1917-1920.
Zaret, K. S. (2008). Genetic programming of liver and pancreas progenitors: lessons for stem-cell differentiation. Nat Rev Genet. 9, 329-340.

1. A method for obtaining a population of hepatic progenitor cells comprising a step of culturing definitive endoderm cells with a culture medium that stimulates hepatic specification.

2. The method according to claim 1, wherein the culture medium that stimulates hepatic specification comprises a retinoic acid receptor (RAR) agonist.

3. The method according to claim 2, wherein the RAR agonist is all-trans retinoic acid (ATRA).

4. The method according to claim 2, wherein the culture medium that stimulates hepatic specification further comprises an FGF family growth factor and an inhibitor of the activin/nodal signaling pathway.

5. The method according to claim 1, wherein:
a) the definitive endoderm cells are cultured with an FGF family growth factor; and
b) the cells cultured in step a) are then cultured with said culture medium that stimulates hepatic specification.

6. The method according to claim 4, wherein the FGF family growth factor is FGF10.

7. The method according to claim 4, wherein the inhibitor of the activin signaling pathway is selected in the group consisting of SB 431542, Lefty-A and Cerberus and derivatives of Lefty-A and Cerberus which inhibit the activin nodal signaling pathway.

8. The method according to claim 1, wherein said definitive endoderm cells are human definitive endoderm cells.

9. The method according to claim 8, wherein said human definitive endoderm cells are obtained from human pluripotent or multipotent stem cells, which are selected in the group consisting of human embryonic stem cells (ES), human pluripotent cells (iPS), umbilical cord blood stem cells, foetal and adult stem cells.

10. A method for obtaining a population of foetal hepatocytes comprising the steps consisting of:
a) producing a population of hepatic progenitor cells by culturing definitive endoderm cells with a culture medium that stimulates hepatic specification,
and
b) differentiating said population of hepatic progenitor cells into foetal hepatocytes.

11. The method according to claim 10, wherein step b) is performed by culturing the hepatic progenitor cells of step a) with a culture medium comprising a FGF family growth factor, an agonist of the EGF signaling pathway and an agonist of the HGF signaling pathway

12. A population of hepatic progenitor cells obtained by culturing definitive endoderm cells with a culture medium that stimulates hepatic specification.

13. A population of foetal hepatocytes obtained by
a) producing a population of hepatic progenitor cells by culturing definitive endoderm cells with a culture medium that stimulates hepatic specification, and
b) differentiating said population of hepatic progenitor cells into foetal hepatocytes.

14. A pharmaceutical composition comprising
a population of hepatic progenitor cells according to claim 12 or a population of foetal hepatocytes according to claim 13 and
a pharmaceutically acceptable carrier or excipient.

15. A method for treating a subject suffering from a hepatic pathology, the method comprising a step of administering to the subject an efficient amount of the population of hepatic progenitor cells as defined in claim 12, or of the pharmaceutical composition as defined in claim 14.

16. A method of producing a chimeric non-human mammal which comprises functional human hepatocytes, comprising the step of injecting into the liver of said non-human mammal human a population of human hepatic progenitor cells according to claim 12 and/or a population of human foetal hepatocytes according to claim 13.


SEQUENCE LISTING (54 sequences)

SEQ ID NO: 1 - DNA, 20 bp, Artificial Sequence
  primer
agtgagaggcaacctggaga

SEQ ID NO: 2 - DNA, 20 bp, Artificial Sequence
  pirmer
acactcggaccacatccttc

SEQ ID NO: 3 - DNA, 27 bp, Artificial Sequence
  primer
ctgctcggagccaccaagagatccatg

SEQ ID NO: 4 - DNA, 25 bp, Artificial Sequence
  primer
atcatctgccacgtgatgctctgca

SEQ ID NO: 5 - DNA, 20 bp, Artificial Sequence
  primer
gggcagatggaagagatcaa

SEQ ID NO: 6 - DNA, 20 bp, Artificial Sequence
  primer
tgcgttcatgaagaagttgc

SEQ ID NO: 7 - DNA, 21 bp, Artificial Sequence
  primer
ctcgaggcttgcccagaccgt

SEQ ID NO: 8 - DNA, 21 bp, Artificial Sequence
  primer
gcgggcttgtcgggatctcag

SEQ ID NO: 9 - DNA, 20 bp, Artificial Sequence
  primer
agaacctgtcacaagctgtg

SEQ ID NO: 10 - DNA, 20 bp, Artificial Sequence
  primer
gacagcaagctgaggatgtc

SEQ ID NO: 11 - DNA, 26 bp, Artificial Sequence
  primer
cctttggcacaatgaagtgggtaacc

SEQ ID NO: 12 - DNA, 24 bp, Artificial Sequence
  primer
cagcagtcagccattcaccatagg

SEQ ID NO: 13 - DNA, 23 bp, Artificial Sequence
  primer
agaccctttgaagtcaagcgacc

SEQ ID NO: 14 - DNA, 25 bp, Artificial Sequence
  primer
ccattgctgaagaccttagtgatgc

SEQ ID NO: 15 - DNA, 21 bp, Artificial Sequence
  primer
ggcagcgaagaagtacaaatc

SEQ ID NO: 16 - DNA, 20 bp, Artificial Sequence
  primer
tcgaacagaatccaactccc

SEQ ID NO: 17 - DNA, 22 bp, Artificial Sequence
  primer
tacagaccctgaagttacccag

SEQ ID NO: 18 - DNA, 21 bp, Artificial Sequence
  primer
taagaagcaatctcctcccga

SEQ ID NO: 19 - DNA, 21 bp, Artificial Sequence
  primer
ggagaaggtcaagagcccgag

SEQ ID NO: 20 - DNA, 22 bp, Artificial Sequence
  primer
agcaaagagtgggtagggacag

SEQ ID NO: 21 - DNA, 20 bp, Artificial Sequence
  primer
tgttggtgtccctttggatt

SEQ ID NO: 22 - DNA, 20 bp, Artificial Sequence
  primer
tcactcaaagcacccaatca

SEQ ID NO: 23 - DNA, 20 bp, Artificial Sequence
  primer
atgacccgtgcctttatcac

SEQ ID NO: 24 - DNA, 20 bp, Artificial Sequence
  primer
tcttggatttgtgggctttc

SEQ ID NO: 25 - DNA, 20 bp, Artificial Sequence
  primer
agaaggcaaacgggtgaacc

SEQ ID NO: 26 - DNA, 20 bp, Artificial Sequence
  primer
gggtcaatgcttctgtgccc

SEQ ID NO: 27 - DNA, 34 bp, Artificial Sequence
  primer
actgaaaaagatgagtatgcctgccgtgtgaacc

SEQ ID NO: 28 - DNA, 34 bp, Artificial Sequence
  primer
cctgctcagatacatcaaacatggagacagcact

SEQ ID NO: 29 - DNA, 20 bp, Artificial Sequence
  primer
agtgagaggcaacctggaga

SEQ ID NO: 30 - DNA, 20 bp, Artificial Sequence
  primer
acactcggaccacatccttc

SEQ ID NO: 31 - DNA, 21 bp, Artificial Sequence
  primer
gatacgccagtgacgaccaga

SEQ ID NO: 32 - DNA, 20 bp, Artificial Sequence
  primer
atcttgctcaactcggcgtt

SEQ ID NO: 33 - DNA, 18 bp, Artificial Sequence
  primer
gggagcggtgaagatgga

SEQ ID NO: 34 - DNA, 22 bp, Artificial Sequence
  primer
tcatgttgctcacggaggagta

SEQ ID NO: 35 - DNA, 23 bp, Artificial Sequence
  primer
tcacagataccagcagcatcagt

SEQ ID NO: 36 - DNA, 19 bp, Artificial Sequence
  primer
gggcatcaccaggcttgta

SEQ ID NO: 37 - DNA, 20 bp, Artificial Sequence
  primer
catggccaagattgaaacct

SEQ ID NO: 38 - DNA, 22 bp, Artificial Sequence
  primer
ttcccatatgttcctgcatcag

SEQ ID NO: 39 - DNA, 18 bp, Artificial Sequence
  primer
cgctccgcttagcagcat

SEQ ID NO: 40 - DNA, 22 bp, Artificial Sequence
  primer
gtgttgcctctatccttcccat

SEQ ID NO: 41 - DNA, 18 bp, Artificial Sequence
  primer
ctcgctggctacccaatt

SEQ ID NO: 42 - DNA, 18 bp, Artificial Sequence
  primer
agaagcgcagttgcttgg

SEQ ID NO: 43 - DNA, 20 bp, Artificial Sequence
  primer
accatgaagtgggtggaatc

SEQ ID NO: 44 - DNA, 20 bp, Artificial Sequence
  primer
tggtagccaggtcagctaaa

SEQ ID NO: 45 - DNA, 20 bp, Artificial Sequence
  primer
ttggcacaatgaagtgggta

SEQ ID NO: 46 - DNA, 20 bp, Artificial Sequence
  primer
aaaggcaatcaacaccaagg

SEQ ID NO: 47 - DNA, 20 bp, Artificial Sequence
  primer
catgcaggactaccccaact

SEQ ID NO: 48 - DNA, 20 bp, Artificial Sequence
  primer
gctacagtggagagggcttg

SEQ ID NO: 49 - DNA, 20 bp, Artificial Sequence
  primer
gcgagagacaggtcaaaacc

SEQ ID NO: 50 - DNA, 20 bp, Artificial Sequence
  primer
agggcgaacattgagagcta

SEQ ID NO: 51 - DNA, 20 bp, Artificial Sequence
  primer
gctggagattaatccggaca

SEQ ID NO: 52 - DNA, 20 bp, Artificial Sequence
  primer
acctgaggctttggattcct

SEQ ID NO: 53 - DNA, 20 bp, Artificial Sequence
  primer
ccgagtccaggatccaggta

SEQ ID NO: 54 - DNA, 19 bp, Artificial Sequence
  primer
ctctgacgccgagacttgg